SEQUENCE LISTING (11 sequences)

SEQ ID NO: 1 - DNA, 29 bp, Synthetic construct
  source 1..29: mol_type = other DNA
  modified_base 23: mod_base = OTHER; note = uracil
aatgatacggcgaccaccgagatctacac

SEQ ID NO: 2 - DNA, 29 bp, Synthetic construct
  modified_base 23: mod_base = OTHER; note = inosine
  source 1..29: mol_type = other DNA
aatgatacggcgaccaccgaganctacac

SEQ ID NO: 3 - DNA, 29 bp, Synthetic construct
  modified_base 23: mod_base = OTHER; note = alkene-thymidine
  source 1..29: mol_type = other DNA
aatgatacggcgaccaccgaganctacac

SEQ ID NO: 4 - DNA, 24 bp, Synthetic construct
  modified_base 22: mod_base = OTHER; note = 8-oxoguanine
  source 1..24: mol_type = other DNA
caagcagaagacggcatacganat

SEQ ID NO: 5 - DNA, 24 bp, Synthetic construct
  source 1..24: mol_type = other DNA
  modified_base 20: mod_base = OTHER; note = 8-oxoguanine
caagcagaagacggcatacnagat

SEQ ID NO: 6 - DNA, 24 bp, Synthetic construct
  modified_base 22: mod_base = OTHER; note = 8-oxoguanine
  modified_base 20: mod_base = OTHER; note = 8-oxoguanine
  source 1..24: mol_type = other DNA
caagcagaagacggcatacnanat

SEQ ID NO: 7 - DNA, 29 bp, Synthetic construct
  source 1..29: mol_type = other DNA
  modified_base 23: mod_base = OTHER; note = Allyl-T
aatgatacggcgaccaccgaganctacac

SEQ ID NO: 8 - DNA, 35 bp, Synthetic construct
  source 1..35: mol_type = other DNA
gctggcacgtccgaacgcttcgttaatccgttgag

SEQ ID NO: 9 - DNA, 35 bp, Synthetic construct
  source 1..35: mol_type = other DNA
cgtcgtctgccatggcgcttcggtggatatgaact

SEQ ID NO: 10 - DNA, 35 bp, Synthetic construct
  source 1..35: mol_type = other DNA
acggccgctaatatcaacgcgtcgaatccgcaact

SEQ ID NO: 11 - DNA, 34 bp, Synthetic construct
  source 1..34: mol_type = other DNA
gccgcgttacgttagccggactattcgatgcagc

(19) United States
(12) Patent Application Publication
(10) Pub. No.: US 2026/0168022 A1
(43) Pub. Date: Jun. 18, 2026

(54) GENOTYPING OR SEQUENCING PLATFORM WITH PASSIVATION LAYER

(71) Applicant: ILLUMINA, INC., San Diego, CA (US)

(72) Inventors: Him Cheng Wong, Singapore (SG); Adithya Ramanitharapandian, Singapore (SG); Kenneth Chong, Singapore (SG); Maxwell Zimmerley, San Diego, CA (US); Leonid Krivitskiy, Singapore (SG); Sara Bakhshi, San Diego, CA (US)

(21) Appl. No.: 19/416,956

(22) Filed: Dec. 11, 2025

Related U.S. Application Data
(60) Provisional application No. 63/733,891, filed on Dec. 13, 2024.

(51) Int. Cl. C12Q 1/6874; B82Y 15/00

(52) U.S. Cl. CPC C12Q 1/6874; B82Y 15/00

(57) ABSTRACT

An example of a genotyping or sequencing platform generally includes a negatively-charged substrate including a plurality of depressions defined therein that are separated by interstitial regions. The genotyping or sequencing platform further includes an exposed passivation layer positioned over an entirety of a surface of the negatively-charged substrate, wherein the exposed passivation layer at least partially fills each depression in the plurality of depressions and at least partially overlies each interstitial region across the entirety of the surface.

### CROSS-REFERENCE TO RELATED APPLICATIONS

This application claims the benefit of U.S. Provisional Application Ser. No. 63/733,891, filed Dec. 13, 2024, the content of which is incorporated by reference herein in its entirety.

### REFERENCE TO SEQUENCE LISTING

The Sequence Listing submitted herewith is hereby incorporated by reference in its entirety. The name of the file is ILI285B_IP-2812-US_Sequence_Listing.xml, the size of the file is 15,088 bytes, and the date of creation of the file is Dec. 4, 2025.

### BACKGROUND

Genotyping is a process that determines differences in an individual's DNA sequence, or genotype, at specific positions in their genome. Genotyping has, at least in part, enabled personalized medicine, which uses an individual's genetic profile to make decisions pertaining to preventative measures, diagnosis, and/or treatments. In the realm of preventative measures and/or diagnosis, genotyping information can identify which disease(s) a patient is most at risk of developing or has, and personalized prevention or treatment plans can be generated. In the realm of treatments, pharmacogenomics (PGx) involves the analysis of genetic variants, such as single nucleotide polymorphisms (SNPs), to identify associations with drug response.

DNA sequencing is a process that determines the order of the four nucleotides in a DNA molecule. Sequencing provides information about an organism's genetic makeup. This information can be useful in identifying genetic diseases, personalized medicine, and determining the cause(s) of various diseases.

### SUMMARY

The genotyping or sequencing platform disclosed herein includes a negatively-charged substrate, which has depressions defined therein. Each depression is to receive an individual particle. For the genotyping platform, the individual particles that are introduced into the depressions are functionalized with sample probes that are designed to query a single base at the 3′ end of each probe. For the sequencing platform, the individual particles that are to be introduced into the depressions are functionalized with primers that are designed for library fragment amplification. In either platform, the negatively-charged substrate includes an exposed passivation layer. The passivation layer at least mitigates an electrostatic repulsion between the functionalized particles and the negatively-charged substrate from occurring, where the electrostatic repulsion would otherwise displace the functionalized particles (i.e., when the passivation layer is omitted). In some instances, the passivation layer may be cationic, and thus can electrostatically attract the functionalized particles (which include negatively-charged probes or primers at their surfaces). Generally, the exposed passivation layer helps to prevent the functionalized particles from being removed from the depressions of the genotyping or sequencing platform, e.g., during washing cycles or use. Preventing removal of the functionalized particles from the depressions of the genotyping or sequencing platform in this manner may improve genotyping or sequencing metrics.

As such, the genotyping or sequencing platform disclosed herein, which includes the exposed passivation layer, may be used to carry out genotyping or sequencing operations, while facilitating the retention of functionalized particles within desired reaction areas.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 depicts a perspective view of an example of the genotyping platform disclosed herein;

FIG. 2 is a cross-sectional view taken along line 2-2 of FIG. 1, illustrating a functionalized particle in a depression of the genotyping platform;

FIG. 3A is a top view of an example of a sequencing platform disclosed herein;

FIG. 3B is an enlarged, and partially cutaway perspective view of an example of the architecture of the sequencing platform of FIG. 3A that includes a plurality of depressions separated by interstitial regions, where an exposed passivation layer is positioned within the depressions and over the interstitial regions;

FIG. 4 is a schematic illustration of an example of a functionalized nanoparticle to be used with the sequencing platform;

FIG. 5A through FIG. 5D collectively show a schematic illustration of various examples of a method of preparing a genotyping platform, where FIG. 5A depicts a negatively-charged substrate having depressions defined therein, FIG. 5B depicts the negatively-charged substrate of FIG. 5A after an exposed passivation layer has been applied thereon, FIG. 5C depicts the structure of FIG. 5B after a plurality of functionalized particles has been loaded into the depressions, and FIG. 5D is an enlarged view that depicts an electrostatic interaction between (i) the exposed passivation layer within the depressions and (ii) functional groups included in (or attached to) the functionalized particles;

FIG. 6 is a graphical representation of the results of an experiment that was performed to analyze the effects of various passivating materials on well/depression occupancy after a wash cycle, where substrate designations (with various passivating conditions/materials) are shown on the x axis and the percentage of the wells occupied (i.e., by the functionalized particles) both before and after the wash cycle is shown on the y axis; and

FIG. 7 includes black-and-white reproductions of four sets of two (top and bottom) optical microscope images that were taken of various genotyping platforms before and after a wash cycle, where the substrate designations (with various passivating conditions/materials) are shown below each set of two images, where the top image in each set of two images represents a genotyping platform before a wash cycle, and where the bottom image in each set of two images represents a genotyping platform after a wash cycle.

### DETAILED DESCRIPTION

The platform disclosed herein may be a genotyping platform or a sequencing platform, depending upon the functionalized particles that are used.

The genotyping platform disclosed herein includes functionalized particles, each of which includes oligonucleotide probes attached to a particle core. The oligonucleotide probes of a given functionalized particle are designed to hybridize selectively to a particular locus in the genome. In some examples, there is a single probe per single nucleotide polymorphism (SNP), and the 3′ end of the probe stops one base short of, i.e., before, the SNP of interest. In other examples, there are two probes per SNP, and the 3′ end of each probe is at the SNP of interest. The two-probe design may be desirable for less common A/T and C/G SNPs.

Within the genotyping platform, the functionalized particles are introduced into depressions that are defined in a negatively-charged substrate. At the surface of the substrate and within each of the depressions is an exposed passivation layer. The exposed passivation layer at least reduces electrostatic repulsion between the functionalized particles and the negatively-charged substrate, and thus improves the retention of the functionalized particles in the depressions during use of the genotyping platform. Retaining the functionalized particles in desired areas of the genotyping platform in this manner may improve genotyping metrics.

The sequencing platform disclosed herein includes functionalized particles, each of which includes a set of oligonucleotide primers attached to a particle core. The two oligonucleotide primers in the set are designed to initiate amplification of a library fragment having end adapters that are respectively complementary to the two oligonucleotide primers.

Similar to the genotyping platform, the functionalized particles of the sequencing platform are introduced into depressions that are defined in a negatively-charged substrate. At the surface of the substrate and within each of the depressions is the exposed passivation layer. The exposed passivation layer at least reduces electrostatic repulsion between the functionalized particles and the negatively-charged substrate, and thus improves the retention of the functionalized particles in the depressions during use of the genotyping platform. Retaining the functionalized particles in desired areas of the sequencing platform in this manner may improve sequencing metrics.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, adjacent, on, etc. are used herein to describe the genotyping platform and/or the various components of the genotyping platform. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

As used herein, the term “attached” refers to the state of two things being joined, fastened, adhered, connected or bound to each other, either indirectly or directly. As an example of an indirect chemical attachment, an aldehyde terminated probe may be attached to a silica particle core that is first functionalized with aminosilane, which is converted into an active monolayer with hydrazine groups (and thus the probe is “indirectly attached” to the silica core). As an example of direct chemical attachment, a probe can be bonded to surface groups of the particle core (and thus the probe is “directly attached” to the core). A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions. Other examples of attachment include mechanical attachment, physical attachment, magnetic attachment, or electrostatic attachment.

The term “depositing,” as used herein, refers to any suitable application technique, which may be manual or automated, and, in some instances, results in modification of the surface properties. Generally, depositing may be performed using vapor deposition techniques, coating techniques, grafting techniques, electroplating techniques, or the like. Some specific examples include chemical vapor deposition (CVD), spray coating (e.g., ultrasonic spray coating), spin coating, dunk or dip coating, doctor blade coating, puddle dispensing, flow through coating/deposition, aerosol printing, screen printing, microcontact printing, inkjet printing, or the like.

As used herein, the term “depression” refers to a discrete recessed feature defined in a substrate and having an opening at a surface of the substrate. The surface opening is at least partially surrounded by interstitial region(s) of the substrate. Depressions can have any of suitable shape at their opening and throughout their depth that can receive the functionalized particles disclosed herein. As examples, the cross-sectional shape may be cylindrical or trapezoidal.

The term “each,” when used in reference to a collection of items, is intended to identify an individual item in the collection, but does not necessarily refer to every item in the collection. Exceptions can occur if explicit disclosure or context clearly dictates otherwise.

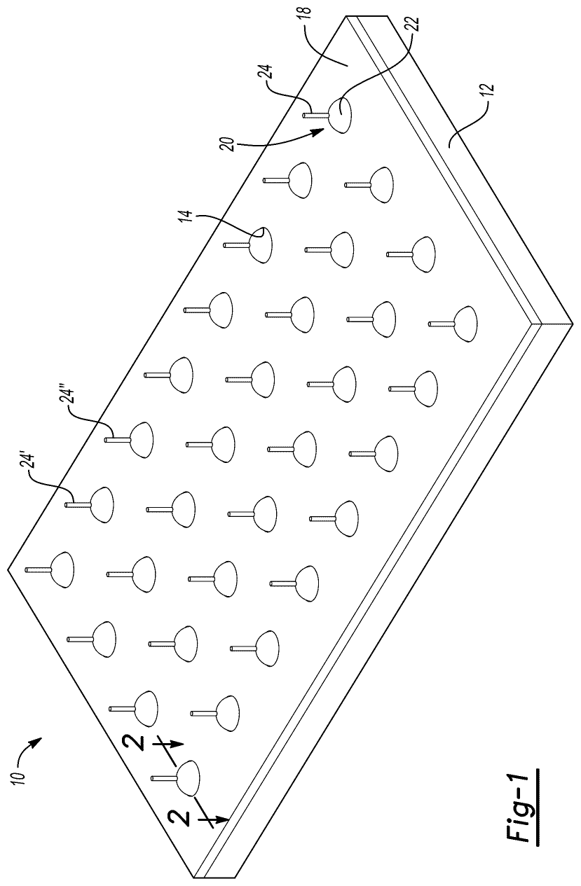

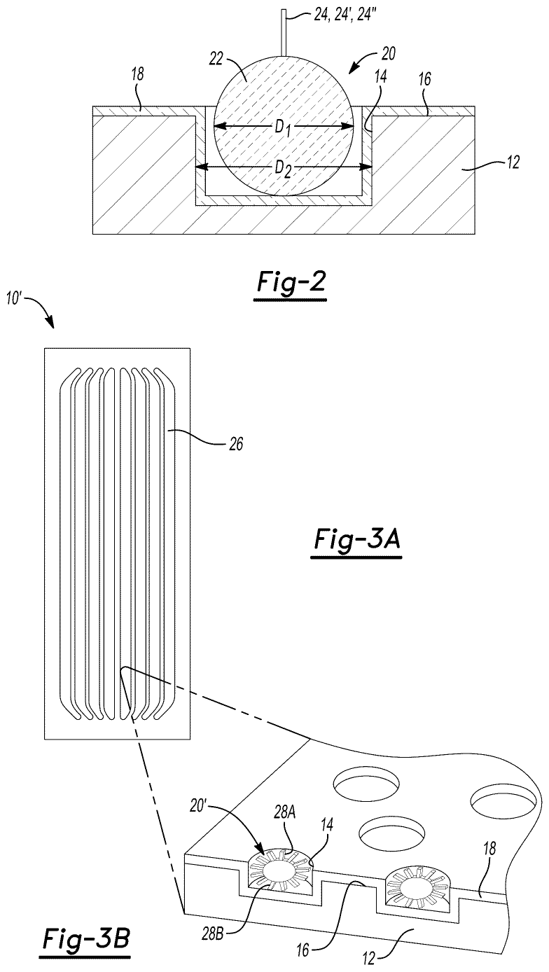

The term “exposed,” as used herein, refers to a material or a structure that has at least one surface that is at least partially open to a surrounding environment. For example, the passivation layer 18 shown in FIG. 1 and FIG. 2 is an “exposed” passivation layer 18 because the passivation layer 18 does not have any continuous layers of materials thereon or thereover (and thus the passivation layer 18 has a surface that is open to a surrounding environment). Further, although the functionalized particles 20 in FIG. 1 and FIG. 2 are shown as being positioned over or on top of the passivation layer 18 within the depressions 14, the functionalized particles 20 are not a continuous layer, and thus the passivation layer 18 is still “exposed” to the surrounding environment.

The term “functionalized particle” refers to i) a particle core, and either ii) a plurality of a genotyping probe attached to the particle core or iii) a plurality of a set of amplification primers attached to the particle core.

As used herein, the term “interstitial region” refers to an area, e.g., of a substrate that separates recessed features, e.g., depressions, from one another. The separation provided by an interstitial region is full separation.

As used herein, the term “negatively-charged substrate” refers to a material that is used to form a genotyping or sequencing platform, where the material includes an inherent property of negative charge (e.g., a cation exchange resin) or that includes or has added thereto negatively-charged surface groups. The negatively-charged substrate is patterned with depressions.

As used herein, a “nucleotide” includes a nitrogen containing heterocyclic base, a sugar, and one or more phosphate groups. Nucleotides are monomeric units of a nucleic acid sequence. In RNA, the sugar is a ribose, and in DNA, the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA). A “labeled nucleotide” is a nucleotide that has at least an optical label attached thereto. Examples of optical labels include any dye that is capable of emitting an optical signal in response to an excitation wavelength.

In some examples, the term “over” may mean that one component or material is positioned directly on another component or material. When one is directly on another, the two are in contact with each other. In FIG. 2, for example, the exposed passivation layer 18 is positioned “directly” over the negatively-charged substrate. In other examples, the term “over” may mean that one component or material is positioned indirectly on another component or material. By indirectly on, it is meant that a gap or an additional component or material may be positioned between the two components or materials. In FIG. 2, for example, the functionalized particles 20 within the depressions 14 are indirectly over the negatively-charged substrate, because the passivation layer 18 is positioned therebetween.

As used herein, the terms “particle core,” and “core” refer to a central material included in a functionalized particle. In some instances, the core is coated with another material that is capable of attaching the oligonucleotide probes or primers thereto. In other instances, the core is comprised of a material that is capable of attaching the oligonucleotide probes or primers thereto.

As used herein, the term “passivation layer” refers to a layer including a material that is capable of blocking, neutralizing, or otherwise suppressing a negative charge at a surface of a resin-based substrate. The term may refer to metals (specific examples are described herein), to silicon dioxide (SiO₂), or to polymeric hydrogel-based materials. In some examples, the passivation layer materials are cationic. The passivation layers disclosed herein are exposed, as the term is defined herein.

As used herein, the term “primer” is defined as a single stranded nucleic acid sequence (e.g., single stranded DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of the core or coating overlying the core. The primer length can be any number of bases long and can include a variety of non-natural nucleotides. In an example, the sequencing primer is a short strand, ranging from 10 to 60 bases, or from 20 to 40 bases. Primers may be attached to a core or to an outer shell of a functionalized nanoparticle.

As used herein, the term “probe” is defined as a single stranded nucleic acid sequence (e.g., single stranded DNA) that is designed to hybridize selectively to a particular locus in the genome, and to query a single base at its 3′ end. In some examples, there is a single probe per single nucleotide polymorphism (SNP), and the 3′ end of the probe stops one base short of, i.e., before, the SNP of interest. In other examples, there are two probes per SNP, and the 3′ end of each probe is at the SNP of interest. The two-probe design may be desirable for less common A/T and C/G SNPs.

### Genotyping Platform

FIG. 1 illustrates an example of the genotyping platform 10 disclosed herein. FIG. 2 illustrates a cross-sectional view of one depression 14 of the genotyping platform 10. Generally, the genotyping platform 10 includes a negatively-charged substrate 12 including a plurality of depressions 14 defined therein that are separated by interstitial regions 16; and an exposed passivation layer 18 positioned over an entirety of a surface of the negatively-charged substrate 12, wherein the exposed passivation layer 18 at least partially fills each depression 14 in the plurality of depressions 14 and at least partially overlies each interstitial region 16 across the entirety of the surface. As depicted in FIG. 1 and FIG. 2, the genotyping platform 10 further includes a plurality of functionalized particles 20 disposed within the plurality of depressions 14.

The negatively-charged substrate 12 is depicted as a single layer base support. While not shown, the negatively-charged substrate 12 may be positioned on another base support. In either instance, the negatively-charged substrate 12 includes the depressions 14 defined at the surface.

Examples of suitable materials for the negatively-charged substrate 12 include siloxanes (e.g., epoxy siloxane), glass, modified or functionalized glass, polymeric materials (including polystyrene and copolymers of styrene and other materials, polypropylene, polyethylene, polyurethanes, polytetrafluoroethylene (such as TEFLON® from Chemours), polyethylene terephthalate (PET), polycarbonate, cyclic olefins/cyclo-olefin polymers (COP) (such as ZEONOR® from Zeon), polyimides, nylon (polyamides), etc.), ceramics/ceramic oxides, aluminum silicate, silicon nitride (Si₃N₄), tantalum pentoxide (Ta₂O₅) or other tantalum oxide(s) (TaOₓ), hafnium oxide (HfO₂), carbon, or the like. Still other suitable negatively-charged substrate 12 materials include polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof. All of the listed materials may be inherently negatively charged or treated to introduce negative charges.

When the negatively-charged substrate 12 is positioned on another base support, any of the listed materials may be stacked, as long as the outermost layer has the depressions 14 defined therein. In one example, the outermost layer may be the polymeric resin layer, and the base support may be silicon nitride.

Many different layouts of the depressions 14 may be used, including regular, repeating, or non-regular patterns. In an example, the depressions 14 are disposed in a hexagonal grid for close packing and improved density. Other layouts may include, for example, rectangular layouts, triangular layouts, and so forth. In some examples, the layout or pattern can be an x-y format in rows and columns. In some other examples, the layout or pattern can be a repeating arrangement of the depressions 14 and interstitial regions 16 (i.e., regions of the substrate surface where depressions 14 are not formed, see FIG. 2). In still other examples, the layout can be a random arrangement of the depressions 14 and the interstitial regions 16.

The layout or pattern may be characterized with respect to the density (number) of the depressions 14 in a defined area. For example, the depressions 14 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. In one example, the density of the depressions 14 ranges from about 725 per mm² to about 22,000 per mm². It is to be further understood that the density can be between one of the lower values and one of the upper values selected from the ranges above, or that other densities (outside of the given ranges) may be used.

The layout or pattern of the depressions 14 may also or alternatively be characterized in terms of the average pitch, or the spacing from the center of one depression 14 to the center of an adjacent depression 14 (center-to-center spacing). The pattern can be regular, such that the coefficient of variation around the average pitch is small, or the pattern can be non-regular in which case the coefficient of variation can be relatively large. In either case, the average pitch can be, for example, about 50 nm, about 0.15 μm (150 nm), about 0.5 μm (500 nm), about 1 μm, about 5 μm, about 10 μm, about 100 μm, or more or less. The average pitch for a particular pattern of depressions 14 can be between one of the lower values and one of the upper values selected from the ranges herein. In an example, the pitch ranges from about 50 nm to about 100 μm. In a specific example, the depressions 14 have a pitch (center-to-center spacing) of about 1.6 μm. While example average pitch values have been provided, it is to be understood that other average pitch values may be used.

The size of each depression 14 is sufficient to receive or spatially accommodate at least a portion of the functionalized particle 20 used in the genotyping platform 10. By “at least a portion” in this context, it is meant that the entire functionalized particle 20 can fit into the depression 14 along its diameter D₂ and that at least half of the functionalized particle 20 can fit into the depression 14 along its depth. As such, the diameter D₂ of the depression 14 is greater than the diameter D₁ of the functionalized particle 20 and the depth of the depression 14 is greater than or equal to 0.5*D₁. The diameter D₂ is representative of the diameter that extends throughout the depth of the depression 14 and of the diameter at the opening of the depression 14. In some examples, the diameter of the functionalized particle 20 ranges from about 200 nm to about 200 μm, and both the depth and the diameter D₂ of the depression 14 are sufficient to accommodate a single functionalized particle 20. For this example, the depth of each depression 14 can range from about 0.1 μm (100 nm) to about 210 μm, and the diameter D₂ of each depression 14 can range from greater than 200.1 nm to about 200.1 μm. In a specific example, both the depth and the second diameter D₂ range from about 1.03 μm to about 1.13 μm. When the diameter D₁ of the functionalized particle 20 is smaller than 200 nm, the size of the depression 14 can be adjusted accordingly so that the functionalized particle 20 tightly fits into the depression 14. For a tight fit, the diameter D₁ of the functionalized particle 20 may be X (some number), and the diameter D₂ of the depression 14 ranges from X to X+5% of X (i.e., ranges from X to X+0.05X).

In the examples disclosed herein, the size of the depression 14 in conjunction with the presence of the passivation layer 18 is sufficient to physically/mechanically immobilize the functionalized particle 20 in the depression 14. This is depicted in FIG. 2. It is to be understood that the size of the depression 14 can be adjusted based on the thickness of the passivation layer 18 and the diameter D₁ of the functionalized particle 20, or that the thickness of the passivation layer 18 can be determined by the diameter D₂ of the depression 14 and the diameter D₁ of the functionalized particle 20 to be introduced thereto.

Additional capture agents are not included in the depressions 14.

The passivation layer 18 may be formed, deposited, or positioned across an entirety of a surface of the negatively-charged substrate 12. The thickness of the passivation layer 18 is such that it aligns, but does not fill, each of the depressions 14, and such that it covers the interstitial regions 16, as shown in FIG. 2. It is to be understood that the thickness of the passivation layer 18 does not interfere with the ability of the depression 14 to receive or spatially accommodate at least a portion of the functionalized particle 20. Thus, the passivation layer 18 thickness is smaller than the depth of the depression 14, and the maximum thickness of the passivation layer 18 is limited so that the functionalized particle 20 can fit into the depression 14. As examples, the passivation layer 18 may have a thickness ranging from about 1 nm to about 50 nm, or from about 5 nm to about 45 nm, or from about 10 nm to about 40 nm, or from about 20 nm to about 30 nm. In specific examples, the passivation layer 18 has a thickness of 5 nm, or of 20 nm. In one example, the passivation layer 18 has a thickness ranging from about 1 nm to about 30 nm. It is to be understood that the thickness of the passivation layer 18 may depend, in part, upon the material(s) used to form the passivation layer 18.

The passivation layer 18 may include any suitable material that is capable of suppressing, neutralizing, or otherwise blocking a negative charge at a surface of (negatively-charged) substrate 12. As such, the passivation layer 18 includes a suitable material that is capable of at least mitigating, and in some instances preventing, electrostatic repulsion between the functionalized particles 20 and the negatively-charged substrate 12. The mitigation or suppression of electrostatic repulsion between the negatively-charged substrate 12 and the negatively-charged moiety of the functionalized particles 20 contributes to the retention of the functionalized particles 20 within the depressions 14 (i.e., by preventing the repulsion from displacing the functionalized particles 20).

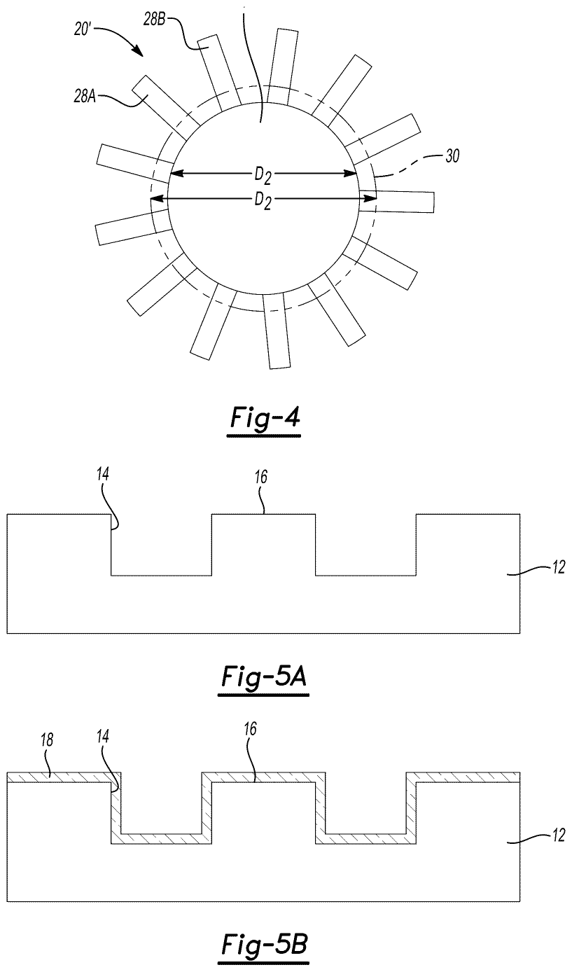

In examples, the passivation layer 18 includes a material that is selected from the group consisting of gold, silver, chromium, silicon dioxide, and a polymeric hydrogel. An example of a suitable polymeric hydrogel is poly (N-(5-azidoacetamidylpentyl)) acrylamide-co-acrylamide, referred to herein as “PAZAM” (described in more detail below in reference to FIG. 3B and FIG. 4).

In some examples, the passivation layer 18 may also experience an electrostatic attraction to a negatively-charged moiety, such as phosphate groups that are included in or attached to nucleotides of the probes 24, 24′, 24″ of the functionalized particles 20 that are positioned in the depressions 14. This interaction between the passivation layer 18 and the negatively-charged moiety contributes to the retention of the functionalized particles 20 within the depressions 14. As such, in some examples of the genotyping platform 10, the probes 24, 24′, 24″ that are attached to the functionalized particles 20 exhibit electrostatic attraction to the exposed passivation layer 18.

The genotyping platform 10 also includes the functionalized particles 20. Each functionalized particle 20 includes a core 22 and a probe 24, 24′, 24″ attached to the core 22.

The core 22 refers to a small body made of a rigid or semi-rigid material. The body can have a shape characterized, for example, as a sphere, oval, microsphere, or other recognized particle shape whether having regular or irregular dimensions. Example materials that are useful for the core 22 include, glass, such as modified or functionalized glass; polymeric materials, such as acrylic, polystyrene or a copolymer of styrene and another material, polypropylene, polyethylene, polybutylene, polyurethane, polyamide, or polytetrafluoroethylene (e.g., TEFLON™ from DuPont); polysaccharides or cross-linked polysaccharides, such as agarose or Sepharose; nitrocellulose; resin; silica; silicon and modified silicon; carbon-fiber; or metal. Example cores 22 include controlled pore glass beads, paramagnetic beads, thoria sol, and Sepharose beads. In one example, the core 22 is a silica bead.

In some examples, the material of the core 22 is capable of binding to the probe 24, 24′, 24″. In other examples, the surface of the core 22 is altered to enable probe 24, 24′, 24″ attachment. For example, the surface of a core 22 can be modified to contain chemically modified sites that are useful for attaching, either-covalently or non-covalently, the probes 24, 24′, 24″. Examples of suitable chemical functional groups include amino groups, carboxy groups, oxo groups and thiol groups, each of which can be used to covalently attach corresponding reactive 5′ terminal groups of the probes 24, 24′, 24″. In another example, the core 22 may also include a coating (not shown) that is selected to bind to a 5′ end functional group of the probe 24. In an example, the core 22 is coated with streptavidin, which can non-covalently bind a biotinylated probe.

The diameter D₁ of the core 22 may range from nanometer sized to micrometer sized. In an example, the diameter D₁ ranges from about 5 nm to about 200 μm. In one example, the core diameter D₁ is about 1000 nm (1 μm).

In the examples disclosed herein, the probes 24, 24′, 24″ are to capture respective target DNA fragment sequences from the human genome. These target sequences can be from any chromosome, e.g., 1-22, X, or Y. In other examples, the probes 24 are to capture respective target DNA fragment sequences from the bovine genome, the maize, genome, the canine genome, the ovine genome, porcine genome, or the shrimp genome.

Because the probes 24, 24′, 24″ are to be used for genotyping, each of the sample probes 24, 24′, 24″ that is attached to a particular core 22 is designed for a specific locus (e.g., SNP) of interest. Thus, each functionalized particle 20 in the genotyping platform 10 enables detection of a unique locus (e.g., SNP) of interest. In other words, each functionalized particle 20 in the plurality of functionalized particles 20 includes unique genotyping probes 24, 24′, 24″.

The probe 24 is a single probe for a single SNP i.e., one probe 24 for both alleles. The 3′ end of the probe 24 stops one base before the locus of interest, and single base extension incorporates one of four nucleotides that confers allele specificity. The sample probes 24 are 50-mer probes and are suitable for non-complementary SNPs (e.g., A to C or G). These probes 24 enable genotyping of most loci in most organisms (e.g., about 84% of known SNPs in the human genome).

The probes 24′ and 24″ are corresponding probes because they are designed for the same SNP. As such, there is one probe 24′ and 24″ for each allele. The 3′ end of each probe 24′, 24″ corresponds with one of two possible bases at the locus of interest. A target DNA fragment is capable of hybridizing to the complementary probe 24′, 24″, and single base extension is enabled due to the hybridization at the 3′ end. Alternatively, the 3′ terminus of the other probe 24′ or 24″ is not able to hybridize to the target DNA fragment, and thus single base extension is not enabled. The mismatched base at the locus of interest will inhibit extension.

It is to be understood that the genotyping platform 10 may include i) functionalized particles 20 that include the probes 24, ii) functionalized particles 20 that include the probes 24′ and functionalized particles 20 that include the probes 24″, or a combination of i) and ii).

Any of the probes 24, 24′, 24″ can be based on the original DNA target strands and/or the complements of the original DNA target strands that are to be hybridized to the probes 24, 24′, 24″. In particular, the probes 24, 24′, 24″ can be designed from the top strand, the bottom strand, the plus stand, or the minus strand. When the A or T in a first unambiguous pair is on the 5′ side of the locus, then the sequence is designated as the top strand sequence. When the A or T in the first unambiguous pair is on the 3′ side of the locus, then the sequence is designated as the bottom strand sequence. The terms plus and minus correspond with the standard designation for all eukaryotic organisms used by HapMap and 1000 Genomes Project. The 5′ end of the (+) strand is at the tip of the short arm (p arm) of the chromosome and the 5′ end of the (−) strand is at the tip of the long arm (q arm).

Each of the probes 24, 24′, 24″ may also include a unique barcode sequence (decoder) portion at its 5′ end. The barcode portion is a nucleotide sequence that may be used to distinguish individual functionalized particles 20. The barcode can be added to the probe 24, 24′, 24″ by methods that physically link or bond the decoder to the probe molecules, e.g., by ligation or transposition through polymerase, endonuclease, transposases, etc.

As mentioned, the probes 24, 24′, 24″ are attached to a respective core 22. While a single sample probe 24, 24′, 24″ is shown attached to each core 22 in FIG. 1 and in FIG. 2, it is to be understood that each core 22 is coated with multiple copies of the respective probes 24, 24′, 24″. The 5′ terminus of each probe probes 24, 24′, 24″ may be modified to allow a coupling reaction with a functional group at or introduced to a surface of the core 22. An example of a 5′ terminal group is biotin.

The probes 24, 24′, 24″ can be attached to the core 22 by sequential addition of monomeric units to synthesize the probes 24, 24′, 24″ on the core 22 in situ. Probes 24, 24′, 24″ can alternatively be synthesized, and then attached to the core 22 using any of a variety of methods known in the art including printing techniques (e.g., ink-jet printing), a spotting technique, a photolithographic synthesis, or printing methods that utilize a mask.

The genotyping platform 10 can retain the functionalized particles 20, respectively, in each of the depressions 14. In the examples disclosed herein, the exposed passivation layer 18 at least mitigates electrostatic repulsion between the functionalized particles 20 and the negatively-charged substrate 12 of the genotyping platform 10. In some instances, the functionalized particles 20 are configured to electrostatically interact with the exposed passivation layer 18. As an example, the unique genotyping probes 24, 24′, 24″ may exhibit electrostatic attraction to the exposed passivation layer 18 when it is cationic. These electrostatic interactions facilitate positional retention of the functionalized particles 20 within the depressions 14 of the genotyping platform 10.

### Sequencing Platform

FIG. 3A illustrates a top view of an example of the sequencing platform 10′ disclosed herein, and FIG. 3B illustrates an example of the architecture within a flow channel 26 of the sequencing platform 10′. The sequencing platform 10′ is similar to the genotyping platform 10 in that it includes a negatively-charged substrate 12 including a plurality of depressions 14 defined therein that are separated by interstitial regions 16; and an exposed passivation layer 18 positioned over an entirety of a surface of the negatively-charged substrate 12, wherein the exposed passivation layer 18 at least partially fills each depression 14 in the plurality of depressions 14 and at least partially overlies each interstitial region 16 across the entirety of the surface.

Any example of the negatively-charged substrate 12 and the exposed passivation layer 18 may be used in the sequencing platform 10′. Any of the dimensions set forth herein for the depressions 14 may be used, and any thickness set forth herein for the passivation layer 18 may be used.

The sequencing platform 10′ may be a flow cell with one or more flow channels 26, which can be open to a surrounding environment or enclosed. An open flow channel 26 is depicted in FIG. 3B, where the depressions 14 are open to the surrounding environment. An enclosed flow channel 26 is formed between the substrate 12 and a lid (not shown) that is attached to the substrate 12 at least at a perimeter of the flow channel 26. The lid may be any material that is transparent to the excitation light that is directed toward the sequencing platform 10′ (e.g., during a sequencing operation). In optical detection systems, the lid may also be transparent to the emissions generated from reaction(s) taking place in the sequencing platform 10′. As examples, the lid may include glass (e.g., borosilicate, fused silica, etc.) or a transparent polymer. A commercially available example of a suitable borosilicate glass is D 263®, available from Schott North America, Inc. Commercially available examples of suitable polymer materials, namely cyclo-olefin polymers, are the ZEONOR® products available from Zeon Chemicals L.P. In some instances, the lid is shaped to form the top of the sequencing platform 10′, and in other instances, the lid is shaped to form both the top of the sequencing platform 10′ as well as sidewalls the flow channel 26 that is formed by the lid and the substate 12. The lid may be bonded to the passivation layer 18, and thus to the substrate 12, using any suitable technique, such as laser bonding, diffusion bonding, anodic bonding, eutectic bonding, plasma activation bonding, glass frit bonding, or others methods known in the art.

As depicted in FIG. 2 and FIG. 3B, the sequencing platform 10′ further includes a plurality of functionalized particles 20′ disposed within the plurality of depressions 14. These functionalized particles 20′ will now be described.

Each of the functionalized particles 20′ includes a core 22′ or 22″ and a set of amplification primers 28A, 28B attached directly to the core 22′ or indirectly to the core 22″.

In one example, the core 22′ is formed of a hydrogel material, and a plurality of the primers 28A, 28B in the set are attached directly to the core 22′. In another example, a hydrogel coating 30 is positioned on a non-hydrogel core 22″, and a plurality of the primers 28A, 28B in the set are attached to the hydrogel coating 30 (and thus indirectly to the core 22″).

The polymeric hydrogel used to form the core 22′ is a semi-rigid polymer that is permeable to liquids and gases. The polymeric hydrogel can swell when liquid (e.g., water) is taken up and that can contract when liquid is removed, e.g., by drying. A hydrogel material may absorb water while not being itself water-soluble.

In some examples, the polymeric hydrogel material is poly (N-(5-azidoacetamidylpentyl) acrylamide-co-acrylamide (PAZAM, as described below) or another of the acrylamide copolymers disclosed herein, poly(ethylene glycol) (PEG)-acrylate, PEG-diacrylate, PEG-amine, PEG-carboxylate, PEG-dithiol, PEG-epoxide, PEG-isocyanate, PEG-maleimide, crosslinked poly(methyl methacrylate) (PMMA), polyvinylpyrrolidone (PVPON), polyvinyl alcohol (PVA), polyethylene oxide-polypropylene oxide block copolymers (PEO-PPO), poly (hydroxyethyl methacrylate) (PHEMA), poly (N-isopropylacrylamide) (PNIPAAm), poly (lactic acid)-poly(ethylene glycol) block copolymers, poly(ethylene glycol)-poly (lactic-co-glycolic acid) block copolymers, poly(acrylic-co-vinylsulfonic acid), poly(acrylamide-co-vinylsulfonic acid), poly (L-aspartic acid), poly (aspartamide), adipic dihydrazide modified or aldehyde modified poly (L-glutamic acid), bisacrylamide, or hydrogels based on one or more of polylysine, starch, agar, agarose, heparin, alginate, alginate sulfate, dextran sulfate, hyaluronan, pectin, carrageenan, gelatin, chitosan, cellulose, and collagen, or combinations or mixtures thereof.

Poly (N-(5-azidoacetamidylpentyl)) acrylamide-co-acrylamide, referred to herein as “PAZAM,” is one example of the hydrogel core 22′. PAZAM and some other forms of the acrylamide copolymer are represented by the following structure (I):

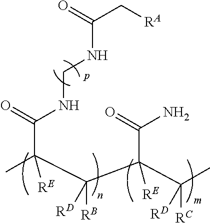

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

The arrangement of the recurring “n” and “m” features in structure (I) are representative, and the monomeric subunits may be present in any order in the polymer structure (e.g., random, block, patterned, or a combination thereof).

The molecular weight of PAZAM and other forms of the acrylamide copolymer may range from about 5 kDa to about 1500 kDa or from about 10 kDa to about 1000 kDa, or may be, in a specific example, about 312 kDa.

In some examples, PAZAM and other forms of the acrylamide copolymer are linear polymers. In some other examples, PAZAM and other forms of the acrylamide copolymer are lightly cross-linked polymers.

In some examples, the gel material may be a variation of structure (I). In one example, the acrylamide unit may be replaced with N, N-dimethylacrylamide

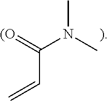

In another example, the acrylamide unit in structure (I) may be replaced with,

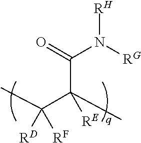

where R^{D}, R^{E}, and R^{F} are each H or a C1-C6 alkyl, and RG and R^{H} are each a C1-C6 alkyl (instead of H as is the case with the acrylamide). In this example, q may be an integer in the range of 1 to 100,000. In another example, the N, N-dimethylacrylamide may be used in addition to the acrylamide unit. In this example, structure (I) may include

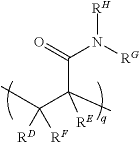

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example, the recurring “n” feature in structure (I) may be replaced with a monomer including a heterocyclic azido group having structure (II):

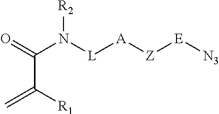

wherein R₁ is H or a C1-C6 alkyl; R² is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the hydrogel core 22′ may include a recurring unit of each of structure (III) and (IV):

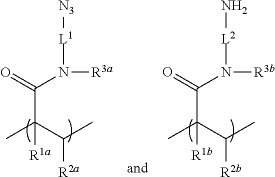

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

Still further examples of suitable polymeric materials for the hydrogel core 22′ include functionalized polysilanes, such as norbornene silane, azido silane, alkyne functionalized silane, amine functionalized silane, maleimide silane, or any other polysilane having functional groups that can attach the oligonucleotide primers 28A, 28B. Other examples of suitable hydrogel materials for the hydrogel coating 14 or the hydrogel core 12′ include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable polymeric hydrogel materials include mixed copolymers of acrylamides and acrylates. A variety of polymer architectures containing acrylic monomers (e.g., acrylamides, acrylates etc.) may be utilized in the examples disclosed herein, such as highly branched polymers, including dendrimers. For example, the monomers (e.g., acrylamide, etc.) may be incorporated, either randomly or in block, into the branches (arms) of a dendrimer.

An example of the dendritic polymeric hydrogel material includes a dendritic core with recurring units of formulas (II) and (III) in the arms extending from the dendritic core. The dendritic core may have anywhere from 3 arms to 30 arms.

The dendritic core may be any multi-functional component that enables a controlled polymerization mechanism, which leads to a defined arm length in the polymer structure and an at least substantially uniform arm length between polymer structures. In an example, the arms of the dendritic core are identical to each other.

The central molecule/compound of the dendritic core may be any multi-functional molecule, such as macrocycles (e.g., cyclodextrins, porphyrins, etc.), extended pi-systems (e.g., perylenes, fullerenes, etc.), metal-ligand complexes, polymeric cores, etc. Some specific examples of the central molecule/compound of the dendritic core include a phenyl group, benzoic acid, pentaerythritol, a phosphazene group, etc.

The dendritic core includes arms that extend from the central molecule/compound. Each arm may include a group that enables the monomers of formula (II) and (III) to be incorporated. In one example, a thiocarbonylthio group is included in each arm, and thus includes a reversible addition-fragmentation chain transfer agent (a RAFT agent). In another example, the dendritic core includes an atom transfer radical polymerization (ATRP) initiator in each arm. In still another example, the dendritic core includes a nitroxide (aminooxy) mediated polymerization (NMP) initiator in each arm.

Functional groups in one or more of the recurring units of the polymeric hydrogel core 22′ are capable of attaching the primers 28A, 28B. These functional groups (e.g., R² in formula (I), NH₂, N₃, etc.) may be located in the side chains of the linear or branched polymeric hydrogel material. As noted, one example of the branched polymeric hydrogel material is a dendrimer, and in an example, the primer-grafting functional groups are located in each of the arms of the dendrimer. These functional groups may be introduced as part of the monomer(s) used in copolymerization. To control the number of primer anchorage points, the monomer bearing the functional group may be increased or decreased. These functional groups may alternatively be introduced after copolymerization.

Polymeric hydrogels may be prepared by cross-linking hydrophilic biopolymers or synthetic polymers or polymerizing suitable monomers and then cross-linking the resulting polymer. Thus, in some examples, the polymeric hydrogel may include a crosslinker. As used herein, the term “crosslinker” refers to a molecule that can form a three-dimensional network when reacted with the appropriate base monomers. Examples of the previously listed hydrogel polymers may include one or more crosslinkers, such as N,N′-bis(acryloyl) cystamine, diamines, dopamine, cysteamine, and aminosilanes. In some examples, a crosslinker forms a disulfide bond in the hydrogel polymer, thereby linking hydrogel polymers.

Any example of the polymeric hydrogel material described herein for the core 22′ may be used as the coating 30 on the non-hydrogel core 22″. Examples of suitable materials for the non-hydrogel core 22″ include magnetic materials (e.g., magnetic FeOₓ, silica coated FeOₓ), polymers (e.g., polytetrafluoroethylene (PTFE), some polyacrylics, polypropylene, polyethylene, polybutylene, polyurethanes, polystyrene and other styrene copolymers), polycaprolactone (PCL), nitrocellulose, silica (SiO₂), silica-based materials (e.g., functionalized SiO₂), carbon, or metals.

As mentioned, the core 22″ supports the hydrogel coating 30. In some examples, each of the plurality of functionalized nanoparticles 20′ further includes silane at a surface of the core 22″; and the polymeric hydrogel coating 30 attached to the silane. Silanization of the core 22″ may be achieved by immersing the core 22″ in a solution including a silane (e.g., trimethoxysilane or another suitable silane) and a suitable organic solvent.

The thickness of the hydrogel coating 30 on the core 22″ ranges from about 10 nm to about 200 nm. The hydrogel coating 30 can be in a dry state or can be in a swollen state, where it uptakes liquid. For example, the 10 nm thickness may represent the hydrogel coating 30 in the fully dry state, and the 200 nm thickness may represent the hydrogel coating 20 in the fully swollen state.

The functionalized particles 20′ have a diameter D₁ as described in reference to the particles 20. When the core 22″ and coating 30 are used, the diameter diameter D₁ is the diameter of the entire particle 20′ including the core 22″ and the coating 30. In examples, the diameter D₁ (e.g., of the functionalized particles 20′) ranges from about 1 nm to about 1000 nm. In other examples, the diameter D₁ ranges from about 225 nm to about 875 nm, or from about 250 nm to about 550 nm, or from about 275 nm to about 325 nm, or from about 290 nm to about 310 nm. These ranges may reflect the diameter D₁ of the functionalized particle 20′ when the hydrogel coating or hydrogel core 22′ is in a swollen state.

As mentioned, the functionalized particles 20′ also include the primers 28A, 28B, which make up a primer set. The polymeric hydrogel coating 30 over the core 22′ or the hydrogel core 22″ provides a surface for attachment of the primers 28A, 28B.

The primer set includes two different primers 28A, 28B, e.g., that are used in sequential paired end sequencing. As examples, the primer set may include P5 and P7 primers, P15 and P7 primers, or any combination of the PA primers, the PB primers, the PC primers, and the PD primers set forth herein. As examples, the primer set may include any two PA, PB, PC, and PD primers, or any combination of one PA primer and one PB, PC, or PD primer, or any combination of one PB primer and one PC or PD primer, or a combination of one PC primer and one PD primer. The P5 and P7 primers have a universal sequence for seeding and/or amplification purposes.

The P5 primer may be any of the following:

P5 #1: 5′ → 3′
(SEQ. ID. NO. 1)
AATGATACGGCGACCACCGAGAUCTACAC
P5 #2: 5′ → 3′
(SEQ. ID. NO. 2)
AATGATACGGCGACCACCGAGAnCTACAC

where “n” is inosine in SEQ. ID. NO. 2; or

P5 #3: 5′ → 3′
(SEQ. ID. NO. 3)
AATGATACGGCGACCACCGAGAnCTACAC

where “n” is alkene-thymidine (i.e., alkene-dT) in SEQ. ID. NO. 3.

The P7 primer may be any of the following:

P7 #1: 5′ → 3′
(SEQ. ID. NO. 4)
CAAGCAGAAGACGGCATACGAnAT
P7 #2: 5′ → 3′
(SEQ. ID. NO. 5)
CAAGCAGAAGACGGCATACnAGAT
P7 #3: 5′ → 3′
(SEQ. ID. NO. 6)
CAAGCAGAAGACGGCATACnAnAT

where “n” is 8-oxoguanine in each of the sequences.

The P15 primer is:

P15: 5′ → 3′
(SEQ. ID. NO. 7)
AATGATACGGCGACCACCGAGAnCTACAC

where “n” is allyl-T (a thymine nucleotide analog having an allyl functionality).

The other primers (PA-PD) mentioned above include:

PA 5′ → 3′
(SEQ. ID. NO. 8)
GCTGGCACGTCCGAACGCTTCGTTAATCCGTTGAG
PB 5′ → 3′
(SEQ. ID. NO. 9)
CGTCGTCTGCCATGGCGCTTCGGTGGATATGAACT
PC 5′ → 3′
(SEQ. ID. NO. 10)
ACGGCCGCTAATATCAACGCGTCGAATCCGCAACT
PD 5′ → 3′
(SEQ. ID. NO. 11)
GCCGCGTTACGTTAGCCGGACTATTCGATGCAGC

While not shown in the example sequences for PA-PD, it is to be understood that any of these primers may include a cleavage site, such as uracil, 8-oxoguanine, allyl-T, etc. at any point in the strand. It is to be further understood that the cleavage sites of the primers 28A, 28B in the primer set are orthogonal to each other (i.e., one cleavage site is not susceptible to a cleaving agent used for the other cleavage site), so that after amplification, forward or reverse strands can be cleaved, leaving the other of the reverse or forward strands for sequencing.

Each of the primers 28A, 28B disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The immobilization of the primers 28A, 28B may be by single point covalent attachment at the 5′ end of the primers 28A, 28B. The attachment will depend, in part, on the functional groups of the hydrogel coating 30 or the hydrogel core 22′. Examples of terminated primers that may be used include an alkyne terminated primer, a tetrazine terminated primer, an azido terminated primer, an amino terminated primer, an epoxy or glycidyl terminated primer, a thiophosphate terminated primer, a thiol terminated primer, an aldehyde terminated primer, a hydrazine terminated primer, a phosphoramidite terminated primer, and a triazolinedione terminated primer. As specific examples, a succinimidyl (NHS) ester terminated primer may be reacted with an amine of the hydrogel coating 30 or the hydrogel core 22′, an aldehyde terminated primer may be reacted with a hydrazine of the hydrogel coating 30 or the hydrogel core 22′, an alkyne terminated primer may be reacted with an azide of the hydrogel coating or the hydrogel core 22′, an azide terminated primer may be reacted with an alkyne or DBCO (dibenzocyclooctyne) of the hydrogel coating 30 or the hydrogel core 22′, an amino terminated primer may be reacted with an activated carboxylate group or NHS ester of the hydrogel coating 30 or the hydrogel core 22′, a thiol terminated primer may be reacted with an alkylating reactant (e.g., iodoacetamine or maleimide) of the hydrogel coating 30 or the hydrogel core 22′, or a phosphoramidite terminated primer may be reacted with a thioether of the hydrogel coating 30 or the hydrogel core 22′. While several examples have been provided, it is to be understood that a functional group that can be attached to the primer 28A, 28B and that can attach to a functional group of the hydrogel coating 30 or the hydrogel core 22′ may be used.

The sequencing platform 10′ can retain the functionalized particles 20′, respectively, in each of the depressions 14. In the examples disclosed herein, the exposed passivation layer 18 at least mitigates electrostatic repulsion between the functionalized particles 20′ and the negatively-charged substrate 12 of the sequencing platform 10′. In some instances, the functionalized particles 20′ are configured to electrostatically interact with the exposed passivation layer 18. As an example, the primers 28A, 28B may exhibit electrostatic attraction to the exposed passivation layer 18 when it is cationic. These electrostatic interactions facilitate positional retention of the functionalized particles 20′ within the depressions 14 of the sequencing platform 10′.

### Genotyping or Sequencing Platform Preparation Method

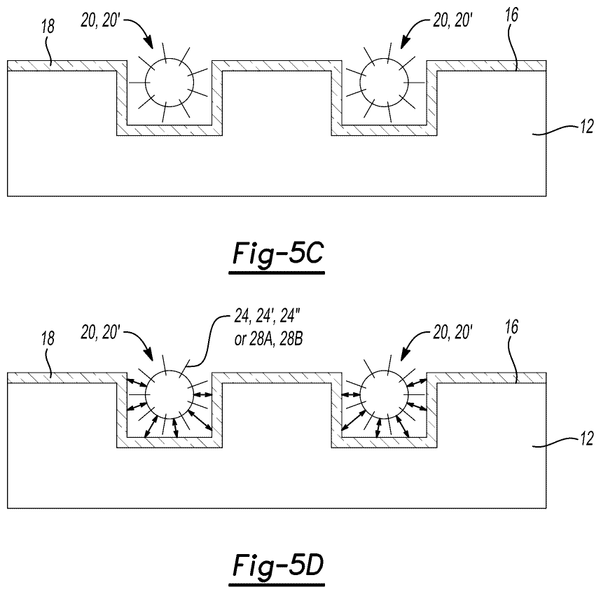

An example of a method of preparing the genotyping platform 10 or sequencing platform 10′ is depicted in FIG. 5A through FIG. 5D. The examples shown in these figures involve applying a passivation material over an entirety of a negatively-charged substrate 12 that includes a plurality of depressions 14 separated by interstitial regions 16, whereby the passivation material at least partially fills each depression 14 in the plurality of depressions 14 and at least partially overlies each of the interstitial regions 16, thereby forming an exposed passivation layer 18.

As shown in FIG. 5A, the negatively-charged substrate 12 includes the depressions 14 defined therein. The depressions 14 may be formed using any suitable patterning technique, such as etching, nanoimprint lithography, nano-injection molding, or photolithography. In a specific example, prior to the applying of the passivation material, the method further comprises defining the plurality of depressions 14 in the negatively-charged substrate 12 using nanoimprint lithography. In this example, the depressions 14 may be formed using a working stamp (not shown). The working stamp, when used, includes a negative replica of the depressions 14 and may be pressed into the negatively-charged substrate 12 while the material of the substrate 12 is soft. Curing of the material of the negatively-charged substrate 12 may then be performed, e.g., via actinic radiation or heat, with the working stamp in place or after removal of the working stamp. Release of the working stamp from the negatively-charged substrate 12 forms the depressions 14 in the negatively-charged substrate 12.

Any suitable materials disclosed herein for the negatively-charged substrate 12 may be used in this example method.

After the negatively-charged substrate 12 has been patterned with the depressions 14, the passivation material may be deposited over an entirety of the surface of the negatively-charged substrate 12, thereby forming the exposed passivation layer 18. The formation of the exposed passivation layer 18 is shown in FIG. 5B. As shown in the figure, the passivation material may be deposited such that the resulting passivation layer 18: (i) is exposed (i.e., is not covered by a continuous layer of another material), (ii) at least partially fills each depression 14, and (iii) at least partially overlies each interstitial region 16 across an entirety of the surface of the substrate 12.

Any suitable material disclosed herein may be used to form the exposed passivation layer 18, such as gold, silver, chromium, silicon dioxide, a polymeric hydrogel, or a combination thereof.

The passivation material may be deposited on the negatively-charged substrate 12 using any suitable deposition technique. In an example, the applying of the passivation material involves spin-coating, sputter-coating, electroplating techniques, chemical vapor deposition, or molecular beam deposition.

The material used to form the exposed passivation layer 18 may be deposited in an amount that is suitable to generate the passivation layer 18 with the thickness set forth herein. In an example, the application of the passivation material over the entirety of the patterned negatively-charged substrate 12 generates a passivation layer 18 having a thickness ranging from about 1 nm to about 30 nm. As mentioned, the thickness is selected so that the functionalized particle 20, 20′ can fit into the depression 14 with the passivation layer 18 therein.

Following the formation of the exposed passivation layer 18, the plurality of functionalized particles 20 or 20′ may be introduced to the surface of the negatively-charged substrate 12 having the exposed passivation layer 18 thereon. This is shown in FIG. 5C. In some examples, the introduction of the functionalized particles 20, 20′ may involve flowing a plurality of functionalized particles 20, 20′ over a surface of the negatively-charged substrate 12, whereby at least some of the functionalized particles 20, 20′ enter the plurality of depressions 14. When the passivation layer 18 is cationic, the functionalized particles 20, 20′ undergo electrostatic attraction to the exposed passivation layer 18.

The primary mechanism for loading and/or retention of the functionalized particles 20, 20′ into or within the respective depressions 14 is via physical immobilization. The exposed passivation layer 18 prevents electrostatic repulsion from occurring between anionic moieties of the functionalized particles 20, 20′ and the negatively-charged substrate 12. In some examples, loading and/or retention may also be enhanced by an electrostatic interaction between (i) negatively-charged moieties, such as phosphate groups, that are included in the functionalized particles 20, 20′, and (ii) a cationic version of the exposed passivation layer 18 within the depressions 14. This electrostatic interaction is shown in FIG. 5D by the two-sided arrow between the exposed passivation layer 18 and the probes 24, 24′, 24″ or primers 28A, 28B of the functionalized particles 20 or 20′. During loading, the functionalized particles 20, 20′ are able to enter the depression 14 due, in part, to external forces that act upon them during the deposition. Those functionalized particles 20, 20′ that do not enter the depressions 14 can be washed from the substrate 12.

### Genotyping Kit

An example of the genotyping kit includes a negatively-charged substrate 12 including a plurality of depressions 14 defined therein separated by interstitial regions 16, and an exposed passivation layer 18 positioned over an entirety of a surface of the negatively-charged substrate 12, wherein the exposed passivation layer 18 at least partially fills each depression 14 in the plurality of depressions 14 and at least partially overlies each interstitial region 16 across the entirety of the surface; and a functionalized particle fluid including a liquid carrier and a plurality of functionalized particles 20 dispersed throughout the liquid carrier, each of the plurality of functionalized particles including a particle core 22 and a plurality of a respective genotyping probe 24, 24′, 24″ attached to the particle core 22. In other example kits, the genotyping platform comes with the functionalized particles 20 present in the depressions 14.

With all the probes 24, 24′, 24″ of genotyping platform 10, marker specificity can be conferred by enzymatic single-base extension to incorporate a generically labeled nucleotide (e.g., nucleotides labeled with dintiophenol, biotin, etc.). Color fluorescent staining enables detection of the incorporated nucleotide. With the single probe 24 per SNP, GT base calling is by single base extension using two-color detection and analysis of the intensity ratio of the two-color signals. With the two probes 24′, 24″ per SNP, GT calling is by allele specific single base extension and analysis of the intensity ratio of the two corresponding probes. Thus, another example of a genotyping kit includes any example of the genotyping platform 10 disclosed herein, a genotyping mixture, and stains.

The genotyping mixture includes labeled nucleotides and a polymerase in a liquid carrier. The nucleotides include the following bases: adenine (tagged with dintrophenol), cytosine (tagged with biotin), guanine (tagged with biotin) and thymine (tagged with dintrophenol). Any polymerase that can accept the nucleotide, and that can successfully incorporate the base of the nucleotide at the 3′ end of the 24, 24′, 24″ may be used. Example polymerases include those polymerases from family A, such as Bsu Polymerase, Bst Polymerase, Taq Polymerase, T7 Polymerase, and many others; polymerases from families B and B2, such as Phi29 polymerase and other highly processive polymerases (family B2), Pfu Polymerase (family B), KOD Polymerase (family B), 90N (family B), and many others; polymerases from family C, such as Escherichia coli DNA Pol III, and many others, polymerases from family D, such as Pyrococcus furiosus DNA Pol II, and many others; polymerases from family X, such as DNA Pol μ, DNA Pol β, DNA Pol σ, and many others. The genotyping mixture may also include a liquid carrier, such as water and/or an ionic salt buffer fluid, e.g., saline citrate at milli-molar to molar concentrations, sodium chloride, potassium chloride, phosphate buffered saline, etc., and other buffers, such as tris (hydroxymethyl)aminomethane (TRIS) or (4-(2-hydroxyethyl)-1-piperazineethanesulfonic acid) (HEPES). The liquid carrier may also include catalytic metal(s) intended for the extension reaction, such as Mg²⁺, Mn²⁺, etc. A single catalytic metal or a combination of catalytic metals may be used, and the total amount may range from about 0.01 mM to about 100 mM.

For the two-color approach mentioned herein, red and green fluorescent dyes may be included in the kit. These dyes are attached, respectively, to anti-DNP and streptavidin.

### Genotyping Method

An example of the method for using the genotyping platform 10 includes generating an amplified DNA sample; and performing a genotyping assay using the genotyping platform 10.

Fragmentation and amplification of the DNA sample that is to be analyzed may be performed using any known method. The amplified DNA sample fragments are then introduced onto the genotyping platform 10, where they will hybridize to corresponding probes 24, 24′, 24″ on the various cores 22.

In one example, an extension reaction is performed at the 3′ ends of the probes 24. The reaction is initiated by introducing the genotyping mixture to the array. For the extension reaction, the genotyping mixture containing the generically labeled nucleotides and the polymerase is introduced into the genotyping platform 10. The temperature of the genotyping platform 10 may be adjusted to initiate the extension reaction. Example temperatures range from about 20° C. to about 70° C. The polymerase enables the extension of the 3′ end of the probe 24, which is adjacent to the locus of interest. The stains can then be introduced, where red or green labeled linkers (e.g., anti-DNP and streptavidin) will attach depending upon the generic label of the incorporated nucleotide. As described, the first extension reaction and the color signal data obtained from this reaction enables one to determine the genotyping status. With the probes 24, each assay genotypes the locus using two color readouts: one color for each allele. The relative intensity of the two colors indicates whether a genotype is heterozygous or homozygous at a particular locus.

In another example, an extension reaction is attempted at the 3′ ends of the probes 24′, 24″. The reaction is initiated by introducing the genotyping mixture to the genotyping platform 10. For the extension reaction, the genotyping mixture containing the generically labeled nucleotides and the polymerase is introduced into the genotyping platform 10. The temperature of the genotyping platform 10 may be adjusted to initiate the extension reaction. Example temperatures range from about 20° C. to about 70° C. The polymerase enables the extension of the 3′ end of the probe 24′ or 24″ that has the DNA sample fragment hybridized at the 3′ end. The corresponding probe 24″ or 24′ that is not fully hybridized at the 3′ end will not undergo the extension reaction. Thus, in this example, the single base extension is allele specific, and the dye label that attaches during staining will be indicative of this allele.

The data derived from any example of the genotyping platform 10 may be used as a discovery tool in order to determine the ethnicity of the individual whose sample is being tested, to identify new single nucleotide polymorphisms in the particular sample, for sample finger printing, sample tracking, or the like.

### Sequencing Kit

An example of the sequencing kit includes a negatively-charged substrate 12 including a plurality of depressions 14 defined therein separated by interstitial regions 16, and an exposed passivation layer 18 positioned over an entirety of a surface of the negatively-charged substrate 12, wherein the exposed passivation layer 18 at least partially fills each depression 14 in the plurality of depressions 14 and at least partially overlies each interstitial region 16 across the entirety of the surface; and a functionalized particle fluid including a liquid carrier and a plurality of functionalized particles 20′ dispersed throughout the liquid carrier, each of the plurality of functionalized particles 20′ including a particle core 22′ or 22″ (with coating 30) and a plurality of a set of primers 28A, 28B attached to the particle core 22′ or 22″. In other example kits, the sequencing platform 10′ comes with the functionalized particles 20′ present in the depressions 14.

The liquid carrier of the functionalized particle fluid may be water and/or an ionic salt buffer fluid, e.g., saline citrate at milli-molar to molar concentrations, sodium chloride, potassium chloride, phosphate buffered saline, etc., and other buffers, such as tris (hydroxymethyl)aminomethane (TRIS) or (4-(2-hydroxyethyl)-1-piperazineethanesulfonic acid) (HEPES).

### Sequencing Method

An example of the method for using the sequencing platform 10′ includes introducing library fragments of a DNA sample to the sequencing platform 10′ at conditions suitable for amplification; and performing a sequencing operation using the sequencing platform 10′.

Library fragments are prepared off-board the sequencing platform 10′. The library fragments/templates may be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the amplification primers 28A, 28B. In some examples, the fragments from a single nucleic acid sample have the same adapters added thereto. The final library templates include the DNA or cDNA fragment and adapters at both ends.

A plurality of library fragments may be introduced to the sequencing platform 10′. One or more library fragments hybridizes, for example, to one of two types of amplification primers 28A, 28B immobilized on the functionalized particle 20′.

Cluster generation may then be performed. In one example of cluster generation, the library fragments are copied from the hybridized primers by 3′ extension using a high-fidelity DNA polymerase. The original library fragments are denatured, leaving the copies immobilized on the functionalized particle 20′. Isothermal bridge amplification or some other form of amplification may be used to amplify the immobilized copies. For example, the copied fragments loop over to hybridize to an adjacent, complementary primer 28A, 28B and a polymerase copies the copied templates to form double stranded bridges, which are denatured to form two single stranded strands. These two strands loop over and hybridize to adjacent, complementary primers 28A, 28B and are extended again to form two new double stranded loops. The process is repeated on each fragment copy by cycles of isothermal denaturation and amplification to create dense clonal clusters. Each cluster of double stranded bridges is denatured. In an example using the primers 28A, 28B, the reverse strand is removed by specific base cleavage, leaving forward fragment strands. Clustering results in the formation of several fragments strands immobilized on the functionalized particle 20′. This example of clustering is referred to as bridge amplification, and is one example of the amplification that may be performed. It is to be understood that other amplification techniques may be used, such as the exclusion amplification (Examp) workflow (Illumina Inc.).

A sequencing primer may then be introduced that hybridizes to a complementary portion of the sequence of the fragments stands. This sequencing primer renders the fragment strand ready for sequencing using an incorporation mix.

The incorporation mix may include a plurality of fully functional nucleotides, the polymerase, and a liquid carrier. The liquid carrier of the incorporation mix may be water and/or an ionic salt buffer fluid, such as saline citrate at millimolar to molar concentrations, sodium chloride, potassium chloride, phosphate buffered saline, etc., and other buffers, such as tris (hydroxymethyl)aminomethane (TRIS) or (4-(2-hydroxyethyl)-1-piperazine ethanesulfonic acid) (HEPES). The liquid carrier may also include catalytic metal(s) intended for the incorporation reaction, such as Mg²⁺, Mn²⁺, Ca²⁺, etc. A single catalytic metal or a combination of catalytic metals may be used, and the total amount may range from about 0.01 mM to about 100 mM.

The fully functional nucleotide includes the nucleotide, a 3′ OH blocking group attached to the sugar of the nucleotide, and a fluorophore attached to the base of the nucleotide. The nucleotide of the FFN may be any nucleotide describe herein.

The nucleotide of the FFN also includes a 3′ OH blocking group attached thereto. The 3′ OH blocking group may be linked to the 3′ oxygen atom of the sugar molecule in the nucleotide. The 3′ OH blocking group may be a reversible terminator that allows only a single-base incorporation to occur in each sequencing cycle. The reversible terminator stops additional bases from being incorporated into a nascent strand that is complementary to the fragment strand. This enables the detection and identification of a single incorporated base. The 3′ OH blocking group can subsequently be removed, enabling additional sequencing cycles to take place at each fragment strand. Examples of different 3′ OH blocking groups include a 3′-ONH₂ reversible terminator, a 3′-O-allyl reversible terminator (i.e., —CH═CHCH₂), and 3′-O-azidomethyl reversible terminator (i.e., —CH₂N₃). Other suitable reversible terminators include o-nitrobenzyl ethers, alkyl o-nitrobenzyl carbonate, ester moieties, other allyl-moieties, acetals (e.g., tert-butoxy-ethoxy), MOM (—CH₂OCH₃) moieties, 2,4-dinitrobenzene sulfenyl, tetrahydrofuranyl ether, 3′ phosphate, ethers, —F, —H₂, —OCH₃, —N₃, —HCOCH₃, and 2-nitrobenzene carbonate.

The nucleotide of the FFN also includes a fluorophore attached to the base of the nucleotide. The fluorophore may be any optically detectable moiety, including luminescent, chemiluminescent, fluorescent, fluorogenic, chromophoric and/or chromogenic moieties. Some examples of suitable optically detectable moieties include fluorescein labels, rhodamine labels, cyanine labels (e.g., Cy3, Cy5, and the like), and the ALEXA® family of fluorescent dyes and other fluorescent and fluorogenic dyes.

The fluorophore may be attached to the base of the nucleotide using any suitable linker molecule. In an example, the linker molecule is a spacer group of formula —((CH₂)₂O)ₙ— wherein n is an integer between 2 and 50. The linker molecule includes a cleavage site.

In one example, the incorporation mix includes a mixture of different FFNs, which include different bases, e.g., A, T, G, C (as well as U or I). It may also be desirable to utilize a different type of fluorophore for the different FFNs. For example, the fluorophores may be selected so that each fluorophore absorbs excitation radiation and/or emits fluorescence at a wavelength that is distinguishable from the other fluorophores. Such distinguishable analogs provide an ability to monitor the presence of different fluorophores simultaneously in the same reaction mixture. In some examples, one of the four FFNs in the incorporation mix may include no fluorophore, while the other three labeled FFNs may include different fluorophore(s).

Any polymerase that can accept the fully functional nucleotide, and that can successfully incorporate the base of the fully functional nucleotide into a nascent strand along the template strand may be used. Example polymerases include those polymerases from family A, such as Bsu Polymerase, Bst Polymerase, Taq Polymerase, T7 Polymerase, and many others; polymerases from families B and B2, such as Phi29 polymerase and other highly processive polymerases (family B2), Pfu Polymerase (family B), KOD Polymerase (family B), 9oN (family B), and many others; polymerases from family C, such as Escherichia coli DNA Pol III, and many others, polymerases from family D, such as Pyrococcus furiosus DNA Pol II, and many others; polymerases from family X, such as DNA Pol μ, DNA Pol β, DNA Pol σ, and many others.

In this example method, any example of the incorporation mix is introduced into the flow channel 26 of the sequencing platform 10′. When the incorporation mix is introduced into the sequencing platform 10′, the mix enters the flow channel 26, and contacts the functionalized particles 20′ where the fragments strands are present.

The incorporation mix is allowed to incubate, and FFNs are incorporated by a polymerase into a nascent strand that is generated along the fragment strand. During incorporation, one of FFNs is incorporated, by a respective polymerase, into one nascent strand that extends one sequencing primer and that is complementary to one of the fragment strands. Incorporation is performed in a template strand dependent fashion, and thus detection of the order and type of FFNs added to the nascent strand can be used to determine the sequence of the fragment strand. Incorporation occurs in at least some of the fragments strands across the depressions 14 during a single sequencing cycle. As such, in at least some of the fragment strands across the sequencing platform 10′, respective polymerases extend the hybridized sequencing primer by one of the FFNs in the incorporation mix.

The incorporated FFNs include the reversible termination property due to the presence of the 3′ OH blocking group, which terminates further sequencing primer extension on the nascent strand once the FFN has been added.

After a desired time for incubation and incorporation, the incorporation mix, including at least some non-incorporated FFNs, may be removed from the sequencing platform 10′ during a wash cycle. The wash cycle may involve a flow-through technique, where a washing solution (e.g., buffer) is directed into, through, and then out of flow channel 26, e.g., by a pump or other suitable mechanism.

Without further incorporation taking place, the most recently incorporated FFNs can be detected through an imaging event. During the imaging event, an illumination system (not shown) may provide an excitation light to sequencing platform 10′. The fluorophore of the incorporated FFNs emit optical signals in response to the excitation light. After imaging is performed, a cleavage mix may then be introduced into the sequencing platform 10′. In this example, the cleavage mix is capable of i) removing the 3′ OH blocking group from the incorporated FFNs, and ii) cleaving the fluorophore from the FFNs. Examples of 3′ OH blocking groups and suitable de-blocking agents/components in the cleavage mix may include: ester moieties that can be removed by base hydrolysis; allyl-moieties that can be removed with NaI, chlorotrimethylsilane and Na₂S₂O₃, or with Hg (II) in acetone/water; azidomethyl which can be cleaved with phosphines, such as tris(2-carboxyethyl)phosphine (TCEP) or tri (hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl, which can be cleaved with nucleophiles such as thiophenol and thiosulfate; tetrahydrofuranyl ether which can be cleaved with Ag (I) or Hg (II); and 3′ phosphate which can be cleaved by phosphatase enzymes (e.g., polynucleotide kinase). Examples of suitable fluorophore cleaving agents/components in the cleavage mix may include: sodium periodate, which can cleave a vicinal diol; phosphines, such as tris(2-carboxyethyl)phosphine (TCEP) or tri (hydroxypropyl)phosphine (THP), which can cleave azidomethyl linkages; palladium and THP, which can cleave an allyl; bases, which can cleave ester moieties; or any other suitable cleaving agent.

Wash(es) may take place between the various fluid delivery steps. The sequencing cycle can then be repeated n times to extend the sequencing primer by n nucleotides, thereby detecting a sequence of length n. In these examples, paired-end sequencing may be used, where the forward strands are sequenced and removed, and then reverse strands are constructed and sequenced.

To further illustrate the present disclosure, an example is given herein. It is to be understood that this example is provided for illustrative purposes and is not to be construed as limiting the scope of the present disclosure.

### Example

To demonstrate the ability of the exposed passivation layer to retain functionalized particles within depressions of a genotyping platform, an experiment was performed utilizing negatively-charged substrates. All of the negatively-charged substrates included a negatively-charged nanoimprint resin material and had depressions defined therein. As described in more detail, several of the negatively-charged substrates also included an exposed passivation layer.

A first control substrate was prepared that did not include an exposed passivation layer (“Control Substrate”). A first experimental substrate was prepared by sputter-depositing gold thereon, which generated an exposed gold passivation layer having a thickness ranging from about 20 nm to about 50 nm (referred to as “Experimental Substrate 1”). A second experimental substrate was prepared by exposing the resin substrate to plasma ashing (i.e., exposure to air plasma), exposing the substrate to a norbornene silane (180 μL silane solution) for a period of about 12 hours and at a temperature of about 60° C., spin-coating a polymeric hydrogel (in a mixture including ethanol and deionized water) onto the silanized substrate, and curing at 60° C. for about 1 hour (referred to as “Experimental Substrate 2”). A third experimental substrate was prepared by sputter-depositing silicon dioxide (SiO₂) thereon for a period of time that was sufficient to generate an exposed silicon dioxide passivation layer having a thickness of about 20 nm (referred to as “Experimental Substrate 3”). A fourth experimental substrate was prepared by sputter-depositing silicon dioxide (SiO₂) thereon for a period of time that was sufficient to generate an exposed silicon dioxide passivation layer having a thickness of about 5 nm (referred to as “Experimental Substrate 4”).

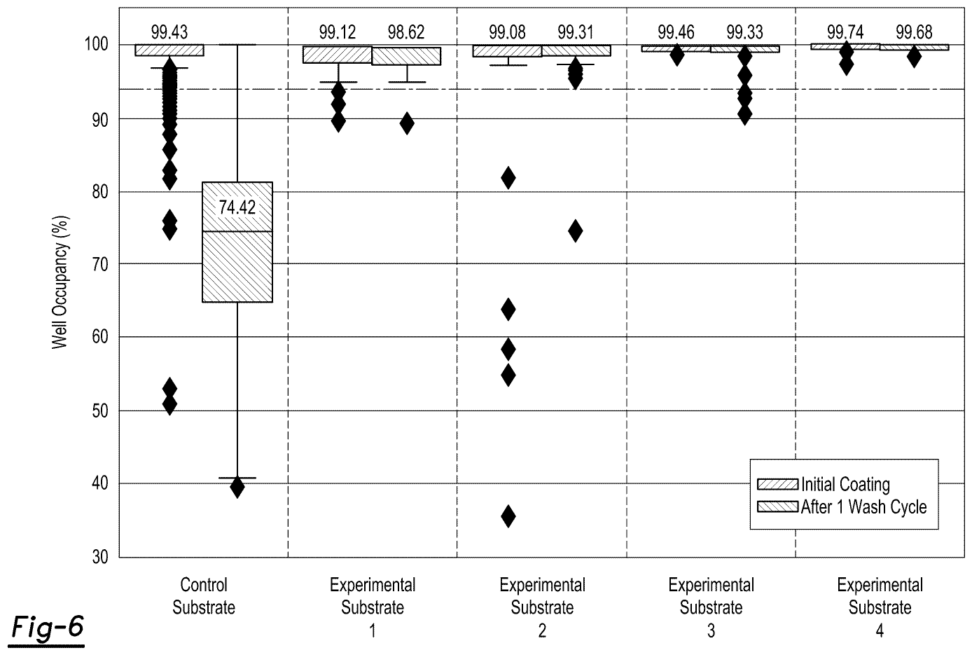

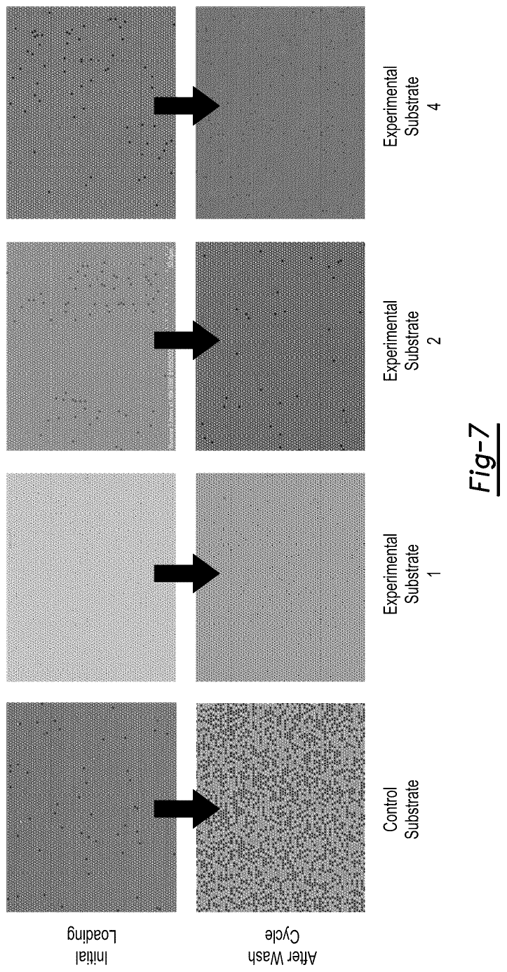

Each of the substrates was loaded with a plurality of functionalized particles having oligonucleotide probes attached thereto, such that the functionalized particles respectively entered individual depressions defined in each of the substrates. An initial measurement of the percentage of the depressions/wells that were occupied by functionalized particles was taken using an optical and scanning electron microscope—these measurements are shown in FIG. 6. As can be seen in the figure, during the initial loading of the functionalized particles into the depressions, over 99% of each of the depressions of each of the substrates was occupied by functionalized particles. An optical microscope image was also taken of the Control Substrate and of the Experimental Substrates 1-4. The images of the Control Substrate and of the Experimental Substrates 1, 2 and 4 are shown at the top of each set of two images in FIG. 7. As can be seen in this figure, for each of the substrates, very few unoccupied depressions can be observed after the initial loading of the functionalized particles (i.e., about 99% of the depressions were occupied).

Atomic force spectroscopy (AFS) was performed using the Control Substrate after functionalized particle loading. During AFS, a small piezoelectric element drives cantilever oscillation near its resonant frequency, which can be shifted depending on the attractive or repulsive forces (force gradient) experienced by the tip at a constant height above the sample surface (during interleave scan). By varying the voltage of the tip, the surface charge of the sample can be deduced by analyzing the shift in phase signal and cantilever resonant frequency. When attractive tip-sample forces were present, the cantilever effectively became softer and vibrated at lower resonant frequency. In contrast, repulsive forces between the tip and sample rendered the cantilever stiffer and increases the resonant frequency. A positive voltage was applied to the tip, and the resonant frequency and phase signal shifted lower, both at the negatively-charged substrate surface and at the functionalized particle surface. From these results, it was deduced that there were attractive forces between the tip (positive) and Control Substrate (negative) and between the tip (positive) and the functionalized particles (negative). These results confirmed both the substrate and the functionalized particles were negatively charged.

Each of the substrates was then exposed to eight cycles of a washing process. During each cycle, the following solutions were sequentially flowed over the respective substrates microfluidically: (i) a strip buffer composed of Al salts and EDTA disodium salt; (ii) a solution of oligonucleotides labelled with fluorescent dye, each designed to hybridize to the probes of the functionalized particles at 40° C.; and (iii) wash/imaging buffer composed of surfactants and NaCl, designed to remove excess, unhybridized oligonucleotides. After the washing process was completed, a second measurement of the percentage of the depressions/wells that were occupied by the functionalized particles was taken using the same method as above. These measurements are also shown in FIG. 6. As can be seen in the figure, after the washing cycles were complete, only about 74% of the depressions/wells of the Control Substrate (which did not include an exposed passivation layer) were occupied by the functionalized particles.

The AFM data indicates that electrostatic repulsion between probes of the functionalized particles and the negatively-charged resin substrate contributed to the decrease in depression/well occupancy that was observed for the Control Substrate. In contrast, after the washing cycles were complete, an average of over 98% of the depressions/wells of Experimental Substrates 1-4 remained occupied by functionalized particles. These results demonstrate the ability of each type of exposed passivation layer to retain functionalized particles within a desired region of a genotyping platform during use (e.g., repeated reagent introduction and removal), such as within the depressions. A second optical microscope image was also taken of the Control Substrate and Experimental Substrates 1, 2, and 4 after the washing processes, and these images are also shown in FIG. 7. As can be seen in the figure, the Control Substrate had many unoccupied depressions/wells after the washing process, whereas each of the Experimental Substrates generally had less unoccupied depressions/wells after the washing cycles, relative to the Control Substrate.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

What is claimed is:

1. A genotyping or sequencing platform, comprising:
a negatively-charged substrate including a plurality of depressions defined therein that are separated by interstitial regions; and
an exposed passivation layer positioned over an entirety of a surface of the negatively-charged substrate, wherein the exposed passivation layer aligns each depression in the plurality of depressions and at least partially overlies each interstitial region across the entirety of the surface.

2. The genotyping or sequencing platform as defined in claim 1, wherein the exposed passivation layer includes a material selected from the group consisting of gold, silver, chromium, silicon dioxide, a polymeric hydrogel, and a combination thereof.

3. The genotyping or sequencing platform as defined in claim 1, further comprising a plurality of functionalized particles disposed within the plurality of depressions.

4. The genotyping or sequencing platform as defined in claim 3, wherein each functionalized particle in the plurality of functionalized particles includes unique genotyping probes.

5. The genotyping or sequencing platform as defined in claim 4, wherein:
the exposed passivation layer is cationic; and
the unique genotyping probes exhibit electrostatic attraction to the exposed passivation layer.

6. The genotyping or sequencing platform as defined in claim 3, wherein each functionalized particle in the plurality of functionalized particles includes a set of amplification primers.

7. The genotyping or sequencing platform as defined in claim 1, wherein the exposed passivation layer has a thickness ranging from about 1 nm to about 30 nm.

8. A method of using the genotyping or sequencing platform as defined in claim 1, comprising:
flowing a plurality of functionalized particles over a surface of the negatively-charged substrate, whereby at least some of the functionalized particles enter the plurality of depressions.

9. A method of preparing a genotyping or sequencing platform surface, comprising:
applying a passivation material over an entirety of a negatively-charged substrate that includes a plurality of depressions separated by interstitial regions defined therein, whereby the passivation material at least partially fills each depression in the plurality of depressions and at least partially overlies each of the interstitial regions, thereby forming an exposed passivation layer.

10. The method as defined in claim 9, wherein the passivation material is selected from the group consisting of gold, silver, chromium, silicon dioxide, a polymeric hydrogel, and a combination thereof.

11. The method as defined in claim 9, wherein the applying of the passivation material involves spin-coating, sputter-coating, electroplating, chemical vapor deposition, or molecular beam deposition.

12. The method as defined in claim 9, wherein prior to the applying of the passivation material, the method further comprises defining the plurality of depressions in the negatively-charged substrate using etching, nanoimprint lithography, nano-injection molding, or photolithography.

13. The method as defined in claim 9, wherein the applying of the passivation material over the entirety of the patterned negatively-charged substrate generates the exposed passivation layer having a thickness that ranges from about 1 nm to about 30 nm.

14. The method as defined in claim 9, further comprising introducing a plurality of functionalized particles to the negatively-charged substrate having the passivation layer thereon, whereby at least some of the plurality of functionalized particles are respectively retained in at least some of the plurality of depressions, and wherein each of the plurality of functionalized particles includes unique genotyping probes.

15. The method as defined in claim 9, further comprising introducing a plurality of functionalized particles to the negatively-charged substrate having the passivation layer thereon, whereby at least some of the plurality of functionalized particles are respectively retained in at least some of the plurality of depressions, and wherein each of the plurality of functionalized particles includes a set of amplification primers.

16. A genotyping kit, comprising:
a genotyping platform, including:
a negatively-charged substrate including a plurality of depressions defined therein separated by interstitial regions; and
an exposed passivation layer positioned over an entirety of a surface of the negatively-charged substrate, wherein the exposed passivation layer at least partially fills each depression in the plurality of depressions and at least partially overlies each interstitial region across the entirety of the surface;
a functionalized particle fluid, including:
a liquid carrier; and
a plurality of functionalized particles dispersed throughout the liquid carrier, each of the plurality of functionalized particles including:
a particle core; and
a plurality of a respective genotyping probe attached to the particle core.

17. The genotyping kit as defined in claim 16, wherein the exposed passivation layer includes a material selected from the group consisting of gold, silver, chromium, silicon dioxide, and a polymeric hydrogel.

18. The genotyping kit as defined in claim 16, wherein the exposed passivation layer has a thickness ranging from about 1 nm to about 30 nm.

19. A sequencing kit, comprising:
a sequencing platform, including:
a negatively-charged substrate including a plurality of depressions defined therein separated by interstitial regions; and
an exposed passivation layer positioned over an entirety of a surface of the negatively-charged substrate, wherein the exposed passivation layer at least partially fills each depression in the plurality of depressions and at least partially overlies each interstitial region across the entirety of the surface;
a functionalized particle fluid, including:
a liquid carrier; and
a plurality of functionalized particles dispersed throughout the liquid carrier, each of the plurality of functionalized particles including:
a particle core; and
a set of amplification primers attached to the particle core.

20. The sequencing kit as defined in claim 19, wherein the exposed passivation layer includes a material selected from the group consisting of gold, silver, chromium, silicon dioxide, and a polymeric hydrogel.

21. The sequencing kit as defined in claim 19, wherein the exposed passivation layer has a thickness ranging from about 1 nm to about 30 nm.
